(19) United States
(12) Patent Application Publication
(10) Pub. No.: US 2006/0105449 A1
(43) Pub. Date: May 18, 2006

(54) Biochip having an electode array on a substrate

(76) Inventors: Franz Larmer, Weil Der Stadt (DE); Heinz Eisenschmid, Ditzingen-Hirschlanden (DE); Michael Stumber, Korntal-Muenchingen (DE); Marko Rocznik, Sindelfingen (DE); Dick Scholten, Stuttgart (DE)

Correspondence Address: KENYON & KENYON LLP, ONE BROADWAY, NEW YORK, NY, 10004, US

(21) Appl. No.: 11/211,928

(22) Filed: Aug. 24, 2005

(30) Foreign Application Priority Data
Sep. 3, 2004 (DE) 10 2004 042 729.1

(51) Int. Cl. C12M 1/34; G01N 27/00

(52) U.S. Cl. 435/287.2; 435/288.4; 422/82.01

(57) ABSTRACT

A biochip for electrical, e.g., capacitive, detection of biochemical molecules has an electrode array on a substrate, the electrodes of the electrode array being each connected through the substrate to an electrically conductive contact surface via an electrical conductor. The contact surfaces are situated on the side of the substrate facing away from the electrode array and form the outermost plane of the substrate on this side.

### FIELD OF THE INVENTION

The present invention relates to a biochip having an electrode array on a substrate for electrically, e.g., capacitively, detecting biochemical molecules, and also relates to a method for manufacturing such a biochip and electrically contacting such a biochip to an electronic analysis system.

### BACKGROUND INFORMATION

Optical methods, fluorescent methods in particular, are nowadays primarily used for detecting biochemical molecules. During a process known as dye marking, fluorescent molecules are chemically appended to the molecules to be detected, whereby the molecules to be detected are labeled with fluorescent molecules. If such molecules are irradiated with UV light or visible light, they absorb the energy from the light and get into an electronically excited state. Via one or multiple transitions from higher energy levels back to lower states, the molecules reach their electronic basic state where they emit the fluorescent light having a certain wavelength. These molecules are therefore referred to as dye molecules. Using a fluorescence microscope, the emitted light of the dye molecule, and thus ultimately the biochemical molecules which were marked with the dye molecules, may be detected.

Although such optical methods exhibit a high sensitivity, they are not optimal for widespread mass application. The equipment necessary for optical detection is relatively complicated and expensive, and to operate it properly requires specially trained personnel. Moreover, it is typically heavy and is only able to be installed stationary in a lab. In particular for tests, in which many, perhaps hundreds or even thousands, of biological samples including biochemical molecules must be tested in simultaneous measurements, there is a need for a simpler detection method.

Possibilities of non-optical detection of biochemical molecules are described in published German patent document DE 199 16 921, for example. This German patent document describes a chip having an electrical sensor array in which ultra-microelectrodes are situated at least in pairs on a planar carrier in grid form, similar to the pattern on a chess board. If a solution containing the analyte molecules, i.e., the biochemical molecules to be detected, is applied to the electrodes, the analyte molecules may then be detected via electrical measurements. The known electrical methods include voltametric and impedimetric detection methods such as redox recycling or impedance measurements. Since certain enzymes must be appended to the analyte molecules in the redox recycling method, unmarked analyte molecules may also be detected using impedance measurements. The impedance between the electrodes changes by the deposition of analyte molecules. Impedance is capacitance less losses, which may be measured using alternating voltage and broken down into a real part and an imaginary part.

In order to execute electrical measurements, in the above-mentioned German patent document the electrodes are guided through direct metallic printed conductors under an insulating layer to individual contact surfaces. The contact surfaces are situated on the top side, i.e., on the same side as the electrodes, and simultaneously on the edge of the chip and offer the possibility of an electrical connection to an external electronic analysis system. Furthermore, additional electronic elements such as transistors, diodes, resistors, and other common electronic components are integrated in certain positions into the chips for individually reading out the individual positions of the sensor array.

Considering that the chips are normally used only once and are subsequently discarded in order to prevent any possibility of corruption due to residues from the previous measurement, there is a need to improve the design of the biochip according to the prior art for a simpler and more efficient operation.

### SUMMARY

The biochip according to the present invention has the advantage that it is easier to operate and less error-prone in comparison to the conventional biochips. In particular, the present invention provides for a simplified and reliable arrangement for contacting the numerous electrodes of the electrode array with an external electronic analysis system. Furthermore, the method according to the present invention makes it possible to manufacture the biochip in a simple and cost-effective manner.

### BRIEF DESCRIPTION OF THE DRAWINGS

FIG. 1 shows a cross-sectional view of a first example embodiment of the biochip according to the present invention.

FIG. 2 shows a cross-sectional view of a second example embodiment of the biochip according to the present invention.

FIG. 3 shows a cross-sectional view of a third example embodiment of the biochip according to the present invention.

FIGS. 4a and 4b show cross-sectional views of a fourth example embodiment of the biochip according to the present invention before and after its completion, respectively.

FIG. 5 shows a cross-sectional view of a fifth example embodiment of the biochip according to the present invention.

FIGS. 6a, 6b and 6c show top views of a first, a second, and a third example embodiment, respectively, of the electrodes of the electrode array.

FIGS. 7a through 7d show cross-sectional views illustrating various steps of an example manufacturing method of the biochip according to the present invention.

### DETAILED DESCRIPTION

The structure of a biochip according to the present invention will be explained in connection with FIG. 1, which shows in a cross-sectional view a biochip 1 having an electrode array 2 on a substrate 5, i.e., a grid-shaped system of electrodes E1, E2 for electrically detecting biochemical molecules. While electrodes E1, E2 are made of gold, or aluminum, or another electrically conductive material, substrate 5 in FIG. 1 is made of silicon. A first insulating layer 3, e.g., Si-oxide, is situated for insulation between substrate 5 made of silicon and electrodes E1, E2. In contrast to the embodiments in the prior art, electrodes E1, E2 of electrode array 2 are each connected through substrate 5 to an electrically conductive contact surface 15 via an electrical conductor 10, contact surfaces 15 being situated on side 20 facing away from electrode array 2 and forming the outermost plane of biochip 1 on this side 20. This side 20, facing away from electrode array 2, is defined as the back side of biochip 1. Electrical conductors 10 and contact surfaces 15 may be made of the same material as electrodes E1, E2, i.e., gold or aluminum in the present example. Furthermore, contact surfaces 15 and substrate 5 are separated from each other by a second insulating layer 35 situated between them. Finally, conductors 10 are also electrically insulated from substrate 5 by a side-wall passivation 36 made of an oxide.

In order to detect biochemical molecules using the above-described biochip 1, these molecules must first be bound, i.e., immobilized, on electrode array 2. It must be ensured that only the sought target substance adheres to individual electrodes E1, E2, and all other molecules are washed away in a cleaning step, for example. A highly selective “key-lock-principle” is therefore used. The core item of all biological substances is DNA chains (deoxyribonucleic acids). In a double helix, two complementary amino acids face each other as basic components—either adenine and thymine or cytosine and guanine. Four different possibilities are available for each position in the chain. In order to bind the biochemical molecules including the DNA strands to electrodes E1, E2, molecules, known as captor molecules, are applied to their surface—shorter DNA chains having an exact complementary sequence to the one which one intends to query. In this context, one also refers to a receptor-ligand system. If the biochemical molecules, which are typically pipetted onto electrodes E1, E2 in the form of sample solutions 4 or applied in a different suitable manner, contain in their DNA chain a section which fits on the captor molecule, then that section adheres to the captor molecule, i.e., it “hybridizes.”

The above-described hybridization reaction may be demonstrated by measuring the change in the electrical impedance between electrodes E1 and E2 caused by it. For this measurement, electrodes E1, E2 are to be electrically connected to an electronic analysis system which is typically situated in an external measuring unit. According to the present invention, the electrodes may be conveniently contacted from the back side, since electrodes E1, E2 are each connected through substrate 5 to a contact surface 15 on the back side of biochip 1 via a conductor 10. This makes it possible to avoid contacting of the numerous contact surfaces 15 to electrical terminals of the external electronic analysis system on the same side of biochip 1 on which electrodes E1, E2 are situated. In order not to accidentally damage the analyte molecules or other sensitive places on the front in the attempt to contact electrode array 2, one must proceed with great care during contacting according to the prior art, which naturally requires more time. In a chip which, as explained above, is only used once and disposed of thereafter, this extra effort is very bothersome in practice. In contrast, according to the present invention, contact surfaces 15 on back side 20 of biochip 1 enable quick and easier contacting with an electronic analysis system.

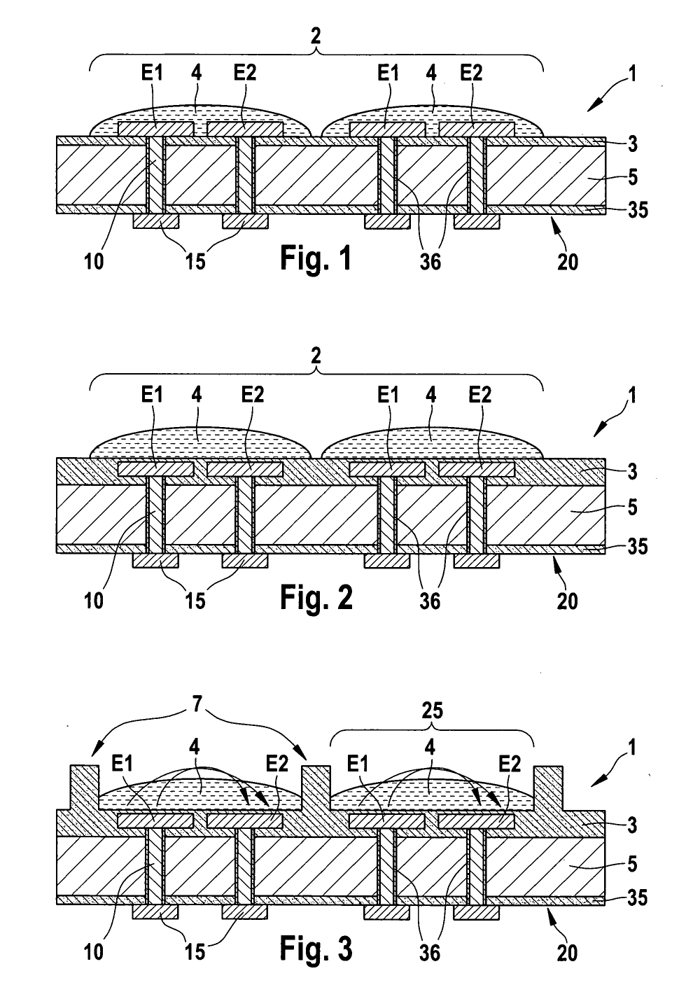

In a second example embodiment of biochip 1, electrodes E1, E2 are not situated on first insulating layer 3 but are rather, as FIG. 2 shows, embedded within first insulating layer 3 and thus insulated from the surroundings. Prior to immobilizing the receptor molecules, a biocompatible, organic coating of insulating layer 3 is applied in this embodiment.

In a third example embodiment, as illustrated in FIG. 3, first insulating layer 3 may have depressions or elevations or another regular structure 7, whereby measuring areas 25 are formed which are separated from one another. Regular structure 7 exactly defines measuring areas 25 as to their form and size and additionally prevents sample solutions 4 from being intermixed by adjacent measuring areas 25. Two electrodes E1, E2 are assigned to each measuring area 25. The arrows between electrodes E1 and E2 in FIG. 3 show the flux distribution characteristic. During a measurement, the lines of flux pass through sample solution 4 including the analyte molecules. Of course, first insulating layer 3 may have a regular structure 7 even when electrodes E1, E2 are not embedded into it, but are situated on it as in FIG. 1.

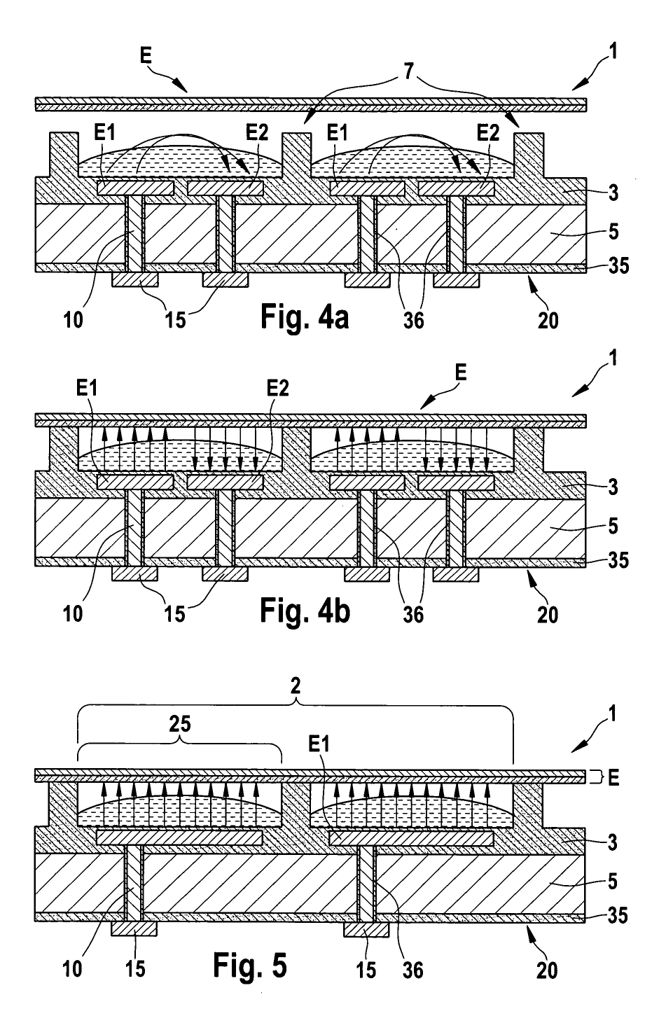

In a fourth example embodiment of biochip 1, a common counter-electrode E, which is positioned on regular structure 7, is additionally provided for electrodes E1, E2. FIG. 4a shows biochip 1 including counter-electrode E before its completion, and FIG. 4b after its completion. In certain cases, depending on the ratio between the dielectric constants of the analyte molecules and first insulating layer 3, it may be advantageous to determine the capacitance via a common counter-electrode E, sample solution 4 including the analyte molecules being situated during the measurement between counter-electrode E and electrodes E1, E2. This makes it possible to increase the spacing between electrodes E1 and E2 and thus decrease the parasitic capacitance caused by electrodes E1 and E2. In addition, the thickness of first insulating layer 3 may be decreased. During the measurement, the potential on counter-electrode E may lie between the potential of electrode E1 and the potential of electrode E2. For clarification, the lines of flux are indicated in FIG. 4b as arrows between electrodes E1, E2 and counter-electrode E. Furthermore, this embodiment has the advantage that a differential readout may be implemented in a particularly easy manner. For this purpose, electrode E1, or the respective location in the case of an embedded electrode, is prepared with an active receptor layer, while electrode E2 or the respective location is prepared with an inactive reference layer. It is important here that the relative dielectric constant of the receptor layer and the reference layer has the same value. An electronic analysis as in a differential capacitor system is now possible.

Furthermore, counter-electrode E may be connected electrically conductively to the biological sample solution, e.g., when potentiometric measurements are intended. It may then be used as a reference electrode.

FIG. 5 shows a fifth example embodiment of biochip 1 in which again one common counter-electrode E is provided for all electrodes E1 of electrode array 2; however, only one electrode E1 is provided for each measuring area 25. A plate capacitor is formed according to the system. The measurement thus determines only the capacitance of the capacitor formed by counter-electrode E and electrode E1, using the solution as the dielectric.

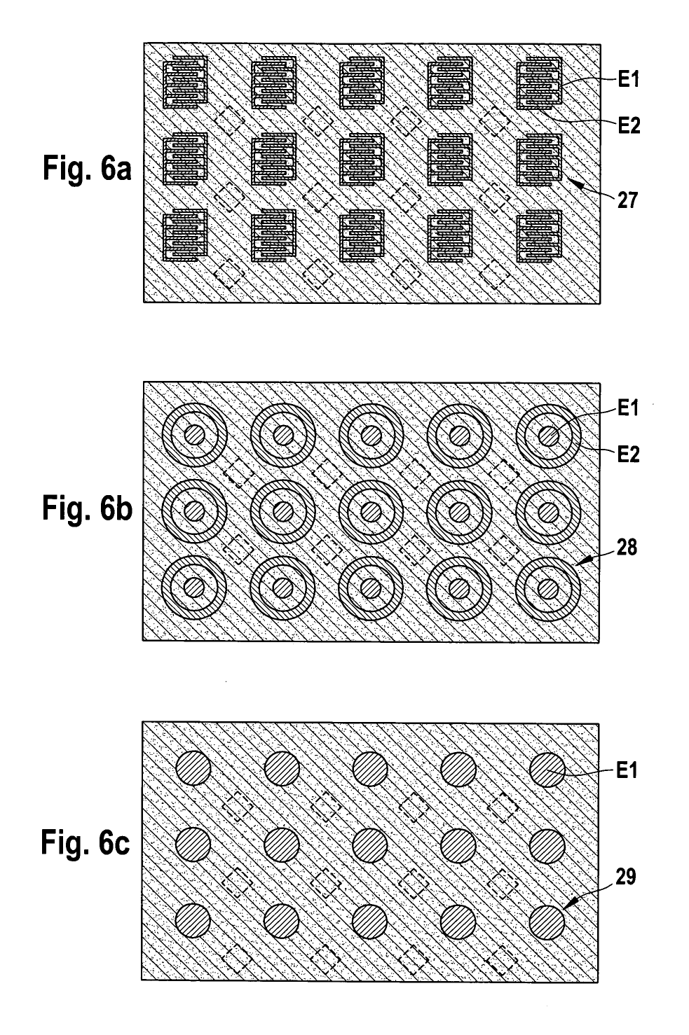

Electrodes E1, E2 may have different shapes. Depending on application and need, electrodes E1, E2 may be designed, for example, as interdigital comb electrodes 27, circular strips 28, or punctiform electrodes 29 as shown (from top-view perspective) in FIGS. 6a, 6b, and 6c.

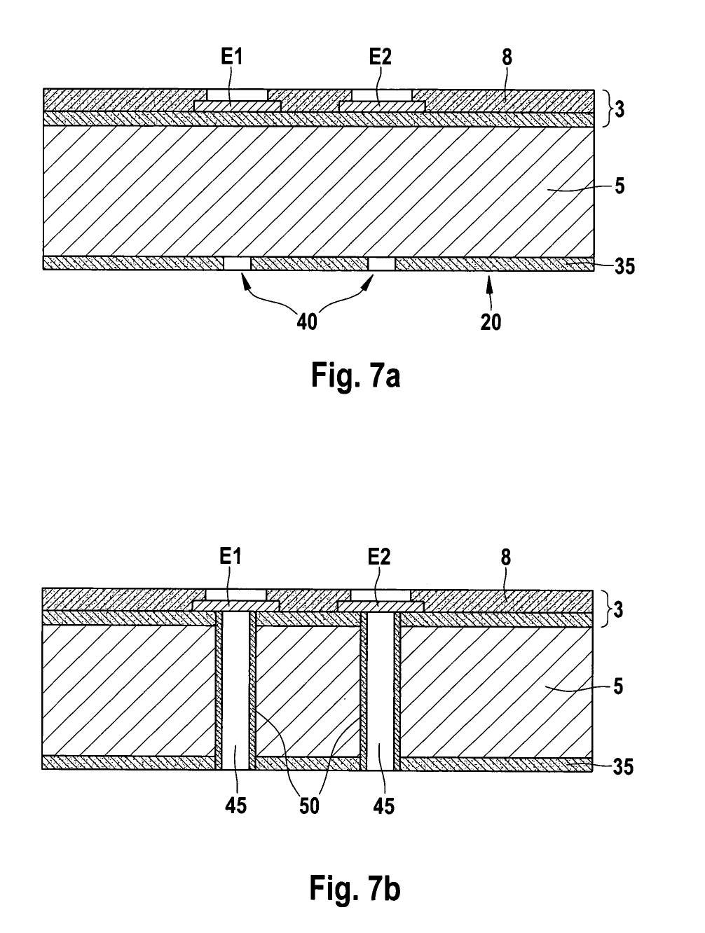

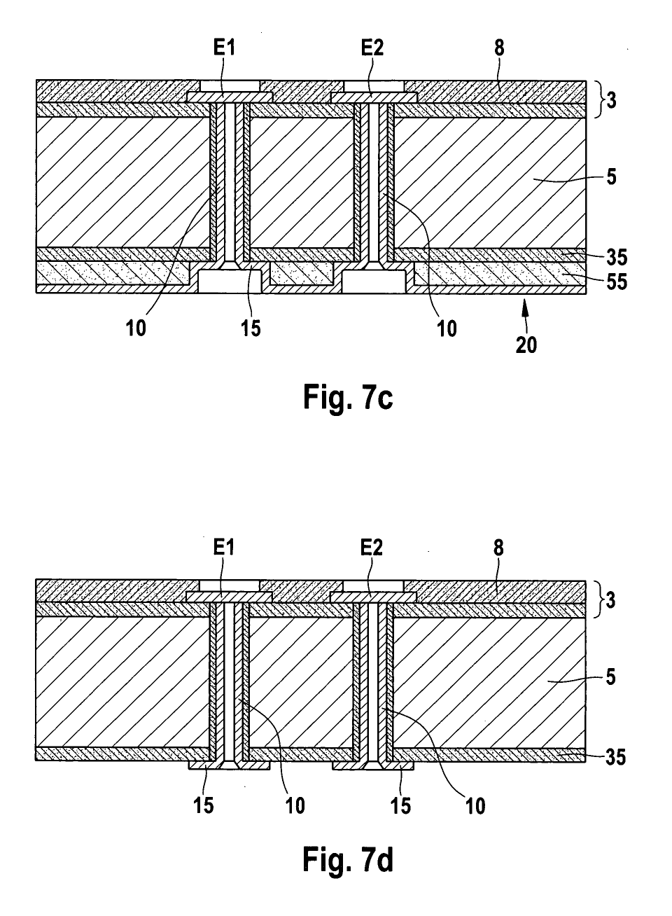

A first example method for manufacturing biochip 1 according to the present invention is now explained based on FIGS. 7a through 7d. Starting material is a substrate 5 made of silicon on which a first insulating layer 3, e.g., made of silicon oxide, is deposited. Furthermore, a metal layer, e.g., made of gold, is applied to first insulating layer 3 and structured to form electrodes E1, E2. First insulating layer 3 may optionally be supplemented, i.e., thickened, by an additional layer 8 and subsequently structured. The material of additional layer 8 is identical to the material of first insulating layer 3 which is thickened by it. On side 20 of the Si substrate facing away from electrodes E1, E2, a second insulating layer 35, e.g., made of silicon oxide, is formed and structured to form apertures 40.

According to FIG. 7b, trenches 45 are formed over apertures 40 through the Si substrate up to electrodes E1, E2. For creating deep trenches 45, an anisotropic etching method (e.g., described in published German patent document DE 42 41 045) may be used, according to which the etching process is carried out separately in separate, alternating sequential etching and polymerization steps. Parts of first insulating layer 3 are also removed in this process. The sidewalls of the trenches are subsequently passivated using an oxide 50.

Finally, as shown in FIG. 7c, a masking layer 55 followed by a metal are deposited on side 20 of the Si substrate facing away from electrodes E1, E2 for forming electrical conductors 10 and contact surfaces 15. The metal may be deposited using a sputtering process or also a galvanic process. The metal may be the same material as electrodes E1, E2.

The completed biochip 1 after removal of masking layer 55 is shown in FIG. 7d. The illustrated method makes it possible to cost-effectively manufacture a biochip 1 including electrodes E1, E2 which are able to be contacted on the back side.

Biochip 1 according to the present invention may have a substrate 5 made not only of silicon, but also of glass or a plastic material. In the field of biosensors, the term “chip” is generally not limited to a silicon substrate. A biochip is generally understood to be a thin carrier on which many different biological samples are situated at certain locations of a grid. Depending on the embodiment and the analysis principle, terms such as “micro-array,” “DNA chip,” “protein chip,” “genome chip,” “gene chip,” or “gene array” are used in the literature in addition to the term “biochip.” The term “biochip” used in this application is to be understood as being equivalent to the above-noted terms.

If a plastic material is selected as substrate 5 of biochip 1, biochip 1 is structured using molding technology. Substrate 5 may optionally enclose part of an enclosure for biochip 1. The enclosure may act as a stable chip carrier and may enable reliable handling and operation, in particular during transport and contacting with an electronic analysis system. Biochip 1 may be metal plated using MID technology (“molded interconnect device” technology), so that, according to the present invention, electrodes E1, E2 of an electrode array 2 are formed which are connected through substrate 5 to an electrically conductive contact surface 15 via an electrical conductor 10, contact surfaces 15 being situated on side 20 of substrate 5 facing away from electrode array 2 and form on this side 20 the outermost plane of substrate 5.

In order to facilitate good and tight contacting of contact surfaces 15 of biochip 1 with an electronic analysis system, the external electronic analysis system may be provided with a system of electrical contact points such as a contact head, a spider-shaped array of needles, or spring pins. The array of the electrical contact points of the electronic analysis system and the contact surfaces 15 of biochip 1 correspond in such a way that joining of both components automatically results in the correct contacts. First, biochip 1 is positioned above the contact points of the electronic analysis system using a housing or a support. The electrical contact is established via controlled vertical displacement of biochip 1 or the array of the electrical contact points of the electronic analysis system. This simple and quick contacting is only made possible by biochip 1 which is contactable on the back side.

What is claimed is:

1. A biochip for electrical detection of biochemical molecules, comprising:
a substrate;
an electrode array positioned on the substrate for the electrical detection of biochemical molecules, wherein the electrode array has a plurality of electrodes;
a plurality of electrical conductors extending through the substrate and corresponding to the plurality of electrodes; and
a plurality of electrically conductive contact surfaces corresponding to the plurality of electrodes, wherein each electrode is connected to a corresponding electrically conductive contact surface by a corresponding electrical conductor extending through the substrate, wherein the plurality of contact surfaces are situated on a side of the substrate facing away from the electrode array and form the outermost plane of the substrate on the side of the substrate facing away from the electrode array.

2. The biochip as recited in claim 1, further comprising:
a first insulating layer positioned on the substrate, wherein the plurality of electrodes are one of situated on the first insulating layer and embedded within the first insulating layer.

3. The biochip as recited in claim 2, wherein the first insulating layer has one of regularly repeating depressions and regularly repeating elevations for defining a plurality of measuring areas, wherein one of adjacent pair of regularly repeating depressions and adjacent pair of regularly repeating elevations defines a measuring area, and wherein the plurality of measuring areas are separated from one another.

4. The biochip as recited in claim 3, further comprising:
a common counter-electrode provided for the plurality of electrodes, wherein the common counter-electrode is positioned on the one of regularly repeating depressions and regularly repeating elevations.

5. The biochip as recited in claim 3, wherein at least one electrode is assigned to each measuring area.

6. The biochip as recited in claim 4, wherein the plurality of electrodes is configured as one of interdigital comb electrodes, circular strips, and punctiform electrodes.

7. The biochip as recited in claim 4, wherein the substrate includes one of silicon, glass, and a plastic material.

8. A method for manufacturing a biochip, comprising:
depositing a first insulating layer made of silicon oxide on a silicon substrate;
depositing a metal layer on the first insulating layer;
structuring the metal layer to form a plurality of electrodes;
structuring the first insulating layer;
forming a second insulating layer on the side of the silicon substrate facing away from the plurality of electrodes;
structuring the second insulating layer to form a plurality of apertures;
forming, using an anisotropic etching process, a plurality of trenches extending from the plurality of apertures, wherein each trench extends through the silicon substrate from a corresponding aperture to a corresponding one of the plurality of electrodes, wherein the anisotropic etching process includes separate, alternating sequential etch and polymerization steps;
passivating sidewalls of the plurality of trenches by an oxide; and
forming a plurality of electrical conductors extending through the substrate and a plurality of electrically conductive contact surfaces with the aid of an interim masking layer by depositing a metal on the side of the silicon substrate facing away from the plurality of electrodes, wherein each electrode is connected to a corresponding electrically conductive contact surface by a corresponding electrical conductor extending through the substrate.

9. A method for manufacturing a biochip including a plurality of electrodes provided on a substrate, a plurality of electrically conductive contact surfaces corresponding to the plurality of electrodes, the method comprising:
providing a substrate made of a plastic material;
structuring the biochip using molding technology; and
metal-plating the biochip using a molded-interconnect-device technology, whereby the plurality of electrodes that are connected to corresponding electrically conductive contact surfaces by corresponding electrical conductors extending through the substrate are formed, and wherein the plurality of contact surfaces are situated on a side of the substrate facing away from the electrode array and form the outermost plane of the substrate on the side of the substrate facing away from the electrode array.

10. A method for electrically contacting a biochip with an electronic analysis system having an array of electrical contact points, wherein the biochip includes a plurality of electrodes provided on a substrate, a plurality of electrically conductive contact surfaces corresponding to the plurality of electrodes, wherein each electrode of the biochip is connected to a corresponding electrically conductive contact surface by a corresponding electrical conductor extending through the substrate, and wherein the plurality of contact surfaces are situated on a side of the substrate facing away from the electrode array and form the outermost plane of the substrate on the side of the substrate facing away from the electrode array, the method comprising:
positioning the biochip laterally above the electrical contact points of the electronic analysis system using one of a housing and a support; and
establishing electrical contact between the contact surfaces of the biochip and the contact points of the electronic analysis system by controlled vertical displacement of one of the biochip and the array of electrical contact points of the electronic analysis system.
